(19) United States
(12) Patent Application Publication
(10) Pub. No.: US 2018/0360424 A1
(43) Pub. Date: Dec. 20, 2018

(54) METHOD AND APPARATUS OF ECHOGENIC CATHETER SYSTEMS

(71) Applicant: CrossBay Medical, Inc., San Diego, CA (US)

(72) Inventors: Matthew Thomas YUREK, San Diego, CA (US); Michael Paul HARTSFIELD, Poway, CA (US); Steven R. BACICH, Half Moon Bay, CA (US)

(73) Assignee: CrossBay Medical, Inc., San Diego, CA (US)

(21) Appl. No.: 16/110,447

(22) Filed: Aug. 23, 2018

Related U.S. Application Data
(63) Continuation of application No. PCT/US2017/020446, filed on Mar. 2, 2017.
(60) Provisional application No. 62/302,194, filed on Mar. 2, 2016.

(51) Int. Cl. A61B 8/08; A61B 8/12

(52) U.S. Cl. CPC A61B 8/481; A61M 25/10; A61B 8/12

(57) ABSTRACT

Methods and apparatuses for utilizing an integrated, automated aerating device for echogenicity are described. The aeration device can have a pressurized vessel to provide echogenic air bubbles independently of fluid delivered for sonohsyterosalpingography. The aeration device can selectively supply a gas in liquid during ultrasound and radiographic procedures for enhanced visualization of the uterine cavity and fallopian tubes.

### CROSS-REFERENCE TO RELATED APPLICATIONS

This application is a continuation of International Application No. PCT/US2017/020446 filed Mar. 2, 2017, which claims priority to U.S. Provisional Application No. 62/302,194, filed Mar. 2, 2016, both of which are incorporated by reference herein in their entireties.

### BACKGROUND

For infertility patients, an assessment of fallopian tube patency is an early evaluation in the patient and couple diagnostic work up. One diagnostic technique is the ultrasound evaluation of tubal patency by the injections of a saline air contrast media that utilizes air bubbles to provide echogenic confirmation of an open fallopian tube. Prior tubal patency assessment systems utilize aeration systems that incorporate verturi components to provide echogenic air bubbles for enhancing ultrasound visualization. These systems require the end user to supply fluid at a flow rate that produces the necessary pressure drop and vacuum to create the aeration effects to pull air bubbles within the fluid media. In clinical operation, intracavity uterine distension pressure supplied by the fluid media needs to exceed the opening cracking pressure of the fallopian tubes. In practice, the requirement to continually add fluid in conjunction with echogenic air bubbles increase patient discomfort due to over distension of the uterine cavity.

Previous aeration systems fail to provide an inexpensive system to build and use since the incorporation of the verturi component typically requires precision engineering, injection molding or machining for the venturi components, and extra assembly steps to build. In addition, the requirement of having two co-linear lumens found in William U.S. Pat. No. 5,211,627, incorporated by reference herein in its entirety, as a representative example of side-by-side lumens, requires the use of a dual collinear lumens; one for the fluid jet and the other for the entrained air bubbles. This lumen configuration requires more space or volume which counteracts the objective of maintaining a low profile device for patient insertion, patient comfort, and ease of handling. Having a system for providing echogenic bubbles during ultrasound procedures that is easier to manufacture, can be manufactured at a lower cost by requiring less components, enables a lower profile, and provides excellent echogenicity within a fluid media is desired.

In addition, having a system for providing echogenic bubbles during ultrasound procedures that is easier to use, provides physicians control over the echogenic air bubbles on demand especially in distended uteri, and enables a more comfortable procedure for the patient by reducing the amount of fluid being injected within the uterine cavity is desired.

### BRIEF SUMMARY OF THE INVENTION

Aeration systems for use in biological target sites and methods of using the same are disclosed.

The aeration system can include an inner tube and an outer tube. At least a portion of the outer tube can overlap the inner tube. The system can include a venturi element within the outer tube. At least a portion of the venturi element can extend beyond a distal end of the inner tube.

The method can include inserting an aerator system into a target site. The aerator system can include an inner tube having an inner lumen, an outer tube having an outer lumen, and a venturi. At least a portion of the inner and outer tubes can be coaxial with one another. At least a portion of the outer lumen can be between the inner tube and the outer tube. The method can include delivering a liquid through the outer lumen and aerating the liquid. Aerating can include delivering a gas through the inner lumen. The method can include directing the aerated liquid to the biological target site.

The aeration system can include an inner tube and an outer tube coaxial with the inner tube. At least a portion of the outer tube can overlap the inner tube.

The method can include inserting an aerator system into a target site. The aerator system can include an inner tube having an inner lumen, an outer tube coaxial with the inner tube, and a venturi. At least a portion of the outer lumen can be between the inner tube and the outer tube. The method can include delivering a liquid through the outer lumen and aerating the liquid. Aerating can include delivering a gas through the inner lumen with a pressurized vessel.

The method can include directing the aerated liquid to the biological target site. The aerator system can include an inner tube and an outer tube coaxial with the inner tube. At least a portion of the outer lumen can be between the inner tube and the outer tube. The system can include a venturi element within the outer tube. At least a portion of the venturi element can extend beyond a distal end of the inner tube. The system can include a pressurized vessel connected to the inner tube.

### BRIEF SUMMARY OF THE DRAWINGS

FIG. 1 is a longitudinal cross-sectional schematic view of a variation of an aeration system.

FIG. 2 is a longitudinal cross-sectional schematic view of a variation of an aeration system.

FIG. 3a illustrates a variation of an aeration system having an inflation balloon, a dual lumen tubing, and a connector.

FIG. 3b is a magnified view of the inflation balloon of FIG. 3a at section 3b-3b.

FIG. 3c is a transparent magnified view of the dual lumen tubing of FIG. 3a at section 3c-3c.

FIG. 3d is a transparent magnified view of the connector of FIG. 3a at section 3d-3d.

FIG. 3e illustrates a variation of an aeration system having an inflation balloon, a dual lumen tubing, and a connector.

FIG. 3f is a magnified view of the inflation balloon of FIG. 3e at section 3f-3f.

FIG. 3g is a transparent magnified view of the connector of FIG. 3e at section 3g-3g.

FIG. 3h is a perspective view of FIGS. 3e-3g.

FIG. 4 is a graph illustrating fluid flow rate with respect to air flow rate for an aeration system having a free-floating air lumen.

FIG. 5a is a perspective view of a variation of an inline eductor insert.

FIG. 5b is a front view of the eductor insert 501 of FIG. 5a.

FIG. 5c is a variation of a longitudinal cross-sectional view of the inline eductor insert of FIG. 5a take along line 5c-5c.

FIG. 5d is a longitudinal cross-sectional view of the distal end of a variation of an aeration system having the inline eductor insert of FIGS. 5a-5c.

FIG. 5e is a perspective view of the aeration system of FIG. 5d.

FIG. 6a is a perspective view of a variation of an inline eductor insert.

FIG. 6b is a rear perspective view of the inline eductor insert of FIG. 6a.

FIG. 6c is a variation of a longitudinal cross-sectional view of the inline eductor insert of FIG. 6a taken along line 6c-6c.

FIG. 6d is a longitudinal cross-sectional view of the distal end of a variation of an aeration system having the inline eductor insert of FIGS. 6a-6c.

FIG. 6e is a magnified view of section A-A of the variation of FIG. 6d.

FIG. 6f is a perspective view of the aeration system of FIG. 6d.

FIG. 7a is a longitudinal cross-sectional view of the distal end of a variation of an aeration system having an inline eductor insert.

FIG. 7b is a perspective view of the aeration system of FIG. 7a.

FIG. 8a is a longitudinal cross-sectional view of a length of a variation of an aeration system.

FIG. 8b is a perspective view of the aeration system of FIG. 8a.

FIG. 9 is a view of a variation of a vessel assembly on the proximal end of an aeration system.

FIG. 10a illustrates a variation of an aeration system having a vessel in an unexpanded configuration and a stopcock for controlling air flow.

FIG. 10b illustrates the vessel of FIG. 10a in an expanded configuration.

FIG. 10c illustrates a variation of the vessel of FIGS. 10a and 10b in an unexpanded configuration.

FIG. 11a is a view of a variation of an aeration system having a gas plug in a closed configuration.

FIG. 11b illustrates the gas plug of FIG. 11a in an open configuration.

FIG. 12 is a graph illustrating air flow versus fluid flow for various aeration systems.

### DETAILED DESCRIPTION

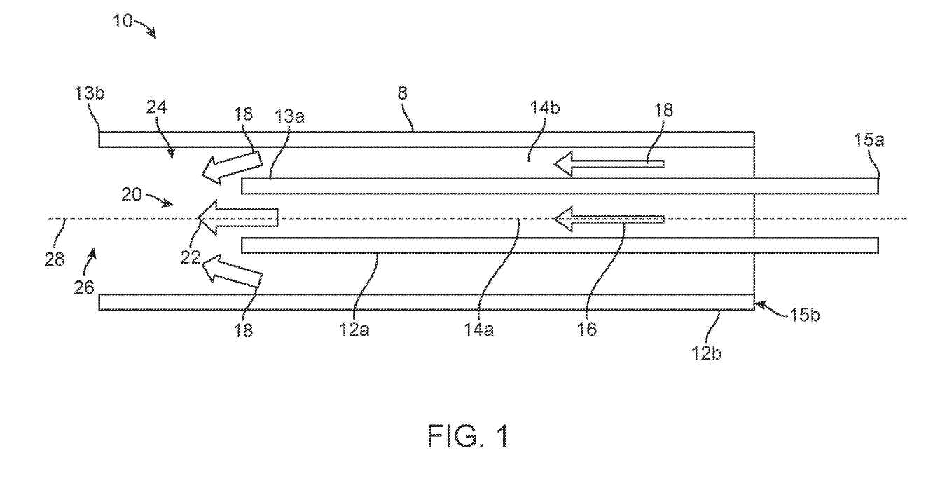

FIG. 1 illustrates that an aeration system 10. can have one or more tubes. The system 10 can have a first tube 12a (also referred to as an inner tube), a second tube 12b (also referred to as an outer tube), and optionally additional tubes (e.g., three tubes, or more than three tubes). The second tube 12b and/or the system 10 can form part of an insertion catheter 8. The first tube 12a can have a first tube inner wall and a first tube outer wall. The second tube 12b can have a second tube inner wall and a second tube outer wall. The first tube 12a can have a first tube proximal end and a first tube distal end. The second tube 12b can have a second tube proximal end and a second tube distal end. The catheter 8 can have a catheter proximal end and a catheter distal end. The first and second tubes 12a, 12b can define first and second tube lumens 14a, 14b, respectively. For example, the inner wall of the first tube 12a can define the first tube lumen 14a and the inner wall of the second tube 12b can define the second tube lumen 14b.

The first tube 12a can be partially or entirely within the second tube lumen 14b of the second tube 12b. For example, FIG. 1 illustrates that a length of the first tube 12a can be within a length of the second tube lumen 14b. For aeration systems having two or more tubes, one or more of the tubes can be within another tube and/or adjacent another tube.

The first and second tube lumens 14a, 14b can be fluid conduits. For example, the first lumen 14a (also referred to as a central lumen) can be a gas lumen/conduit and the second lumen 14b (also referred to as an outer lumen) can be a liquid lumen/conduit, or vice versa. The first lumen 14a can be a conduit for a gas (e.g., air) supply that can be entrained within a fluid media. The second lumen 14b can be a conduit for fluid delivery (e.g., liquid delivery).

FIG. 1 illustrates that fluids 16, 18 can flow through the first and second lumens 14a, 14b. For example, a gas 16 can flow through the first lumen 14a and a liquid 18 can flow through the second lumen 14b. Conversely, the system 10 can be configured with the central lumen 14a as the conduit for the liquid 18 and the outer lumen 14b as the conduit for the gas 16. The gas 16 can be a single gas or a combination of gases. The liquid 18 can be a single liquid or a combination of liquids. The gas 16 can be, for example, carbon dioxide, nitrogen, oxygen, steam (water vapor), or combinations thereof (e.g., air). The liquid 18 can be, for example, saline, saline solution, water, or combinations thereof.

The liquid 18 (e.g., in the second lumen 14b, in the second tube 12b) can be an aerated or non-aerated liquid. The gas 16 can be injected to a biological target site by a physician or operator operating the system 10. The liquid 18 can be injected to a biological target site by a physician or operator operating the system 10.

The system 10 can mix the gas 16 and the liquid 18 to create an aerated liquid 22 having gas bubbles. The gas 16 can be mixed with the liquid 18 (or vice versa), for example, within the catheter 8 and/or within the system 10. The gas 16 can be entrained within the liquid 18, for example, within the catheter 8 and/or within the system 10. The gas 16 and the liquid 18 can be mixed at a distal end of the catheter 8.

FIG. 1 illustrates that the system 10 can have a throat 20 (also referred to as a venturi), an outlet channel 24, and an outlet port 26. As shown, the throat 20, the outlet channel 24, and the outlet port 26 can be at a distal end of the system 10. The throat 20 can be between a distal terminal end 13a of the first tube 12a and a distal terminal end 13b of the second tube 12b, or anywhere along the length of the first and/or second tubes 12a, 12b (e.g., anywhere along the length of the first and/or second tubes 12a, 12b between their respective terminal ends). The throat 20 can decrease the pressure at the distal end 13a of the first lumen 14a by changing the fluid velocity in the system 10. The decrease in pressure can pull the gas 16 into the first lumen 14a (e.g., at a first end of the first tube 12a, at a proximal end of the first tube 12a) and into the liquid 18 (e.g., at a second end of the first tube 12a, at a distal end of the first tube 12a). This can create an aerated liquid 22 that can be delivered to a biological target site. In this way, the throat 20 can facilitate the mixing of the fluids 16, 18. The mixing of the fluids 16, 18 can aerate the fluid 18 to produce the aerated liquid 22 (i.e., the aerated liquid 22 can be a combination/mixture of the fluids 16, 18). If the liquid 18 is already partially aerated, the mixing of the fluids 16, 18 can further aerate the liquid 18 to produce the aerated liquid 22.

As used herein, the term “aerate” can include adding a volume of gas to a fluid, increasing the volume of gas in the fluid, and/or increasing the surface area of the volume of gas in the fluid. For example, gas can be added to the fluid, the number of gas bubbles in the fluid can be increased and/or decreased, and/or the size of gas bubbles in the fluid can be increased and/or decreased. The term “aerate” can include removing a volume of gas from the fluid, decreasing the volume of gas in the fluid, and/or decreasing the surface area of the volume of gas in the fluid. For example, gas can be removed from the fluid, the number of gas bubbles in the fluid can be increased and/or decreased, and/or the size of gas bubbles in the fluid can be increased and/or decreased.

The aerated liquid 22 can flow though the outlet channel 24 before exiting the system 10 through the outlet port 26. The outlet port 26 can be at the tip and/or distal end of the catheter 8. The system 10 can have multiple outlet ports 26. The second tube 12b can define the outlet channel 24 and/or the outlet port 26. The distal terminal end 13a of the first lumen 14a can be at a specific dimensional location relative to the outlet channel 24 and/or the outlet port 26. The outlet channel 24 can have a first end and a second end. The first end of the outlet channel 24 can coincide with where the first lumen 14a terminates (e.g., at the distal terminal end 13a of the first tube 12a), and the second end of the outlet channel 24 can coincide with the outlet port 26 (e.g., at the distal terminal end 13b of the second tube 12b). Other arrangements are also appreciated. For example, the distal terminal ends 13a, 13b of the first and second tubes 12a, 12b can coincide or substantially coincide such that at least a portion of the gas 16 and the liquid 18 mixes outside of the system 10.

FIG. 1 illustrates that the first and second tubes 12a, 12b can be concentrically or coaxially aligned along an axis 28 (e.g., a longitudinal axis). The first and second lumens 14a, 14b can be concentrically or coaxially aligned along the axis 28. For example, the first and second lumens 14a, 14b can be concentrically or coaxially aligned within a wall of the second tube 12b (e.g., within an inner and/or outer surface of a wall of the second tube 12b). Other alignments of the first and second tubes 12a, 12b and/or the first and second lumens 14a, 14b are also appreciated. For example, the first and second tubes 12a, 12b and/or the first and second lumens 14a, 14b can be non-concentrically or non-coaxially aligned along an axis (e.g., along a longitudinal axis of the first tube 12a and/or the second tube 12b). As another example, the first and second tubes 12a, 12b and/or the first and second lumens 14a, 14b can be concentrically or coaxially aligned along one or more portions of an axis and/or can be non-concentrically or non-coaxially aligned along one or more portions of an axis.

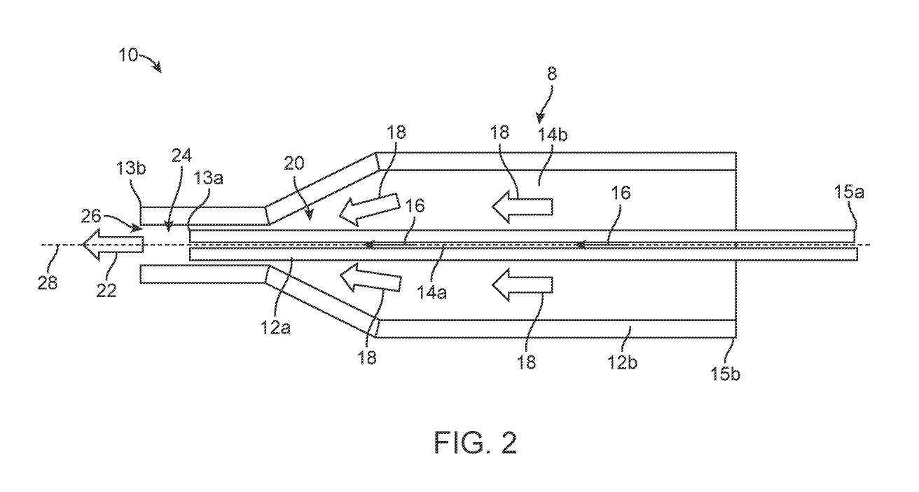

FIG. 2 illustrates that the throat 20 of FIG. 1 can be tapered. As shown, the throat 20 can be between a distal terminal end 13a and a proximal terminal end 15a of the first tube 12a and can be between a distal terminal end 13b and a proximal terminal end 15b of the second tube 12b. Other arrangements are also appreciated, including anywhere along the length of the first and/or second tubes 12a, 12b. The throat 20 can taper from a first cross-sectional area to a second cross-sectional area. The first cross-sectional area can be greater than the second cross sectional area. For example, the wall of the second tube 12b can change in diameter (e.g., internal diameter) at the throat 20. As shown, the wall of the second tube 12b can decrease from a first diameter to a second diameter. The tapered throat 20 can be manufactured into the catheter tubing by drawing down the tubing in manufacturing, in the tubing extrusion process, employing two tubing components of different internal diameters that are assembled together, or combinations thereof.

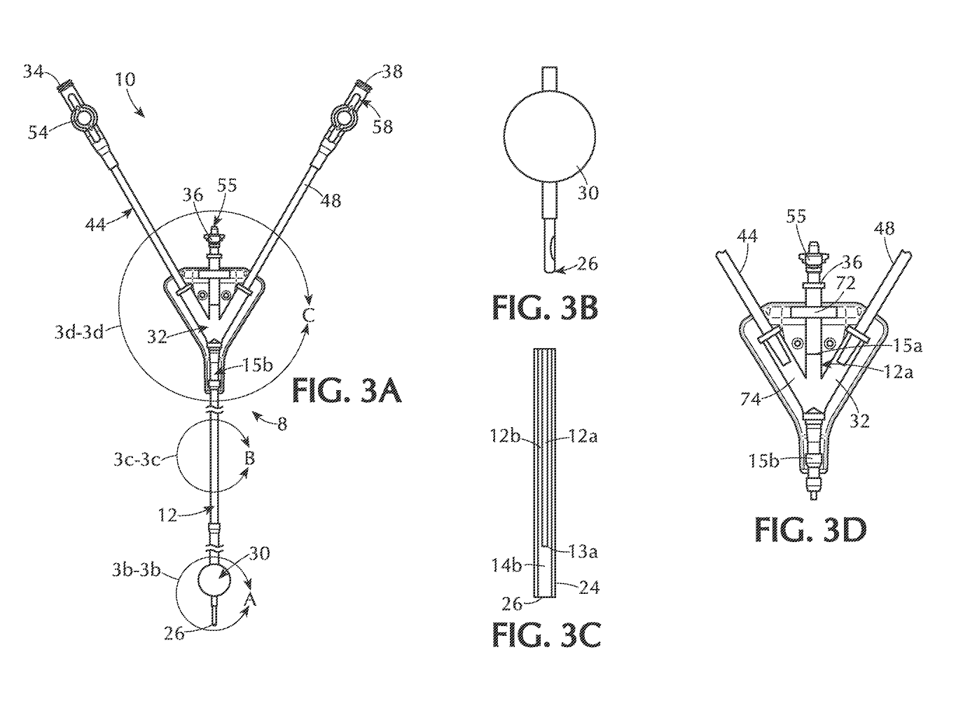

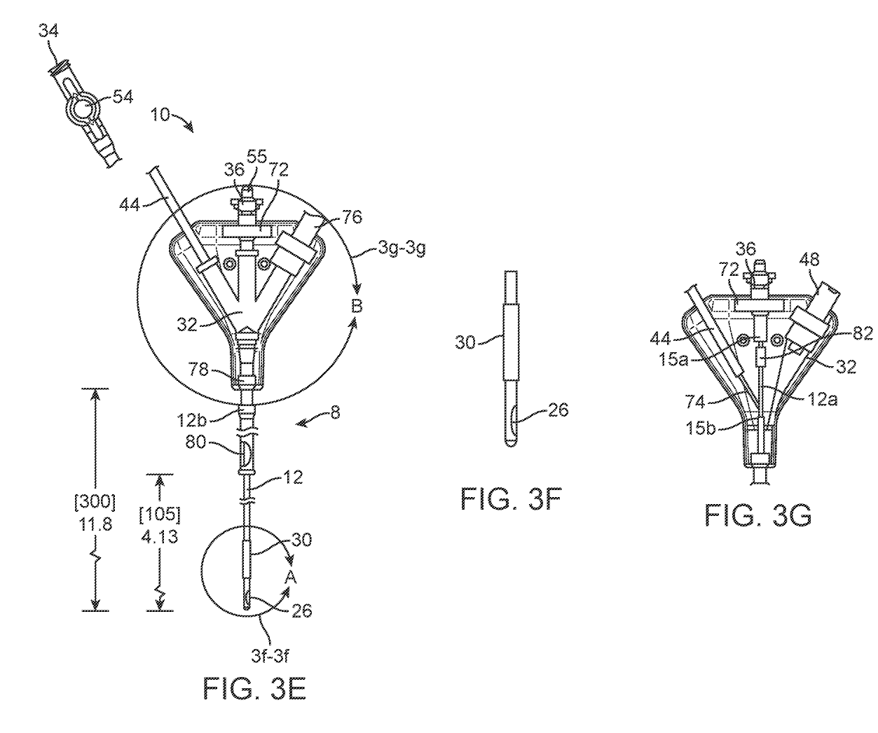

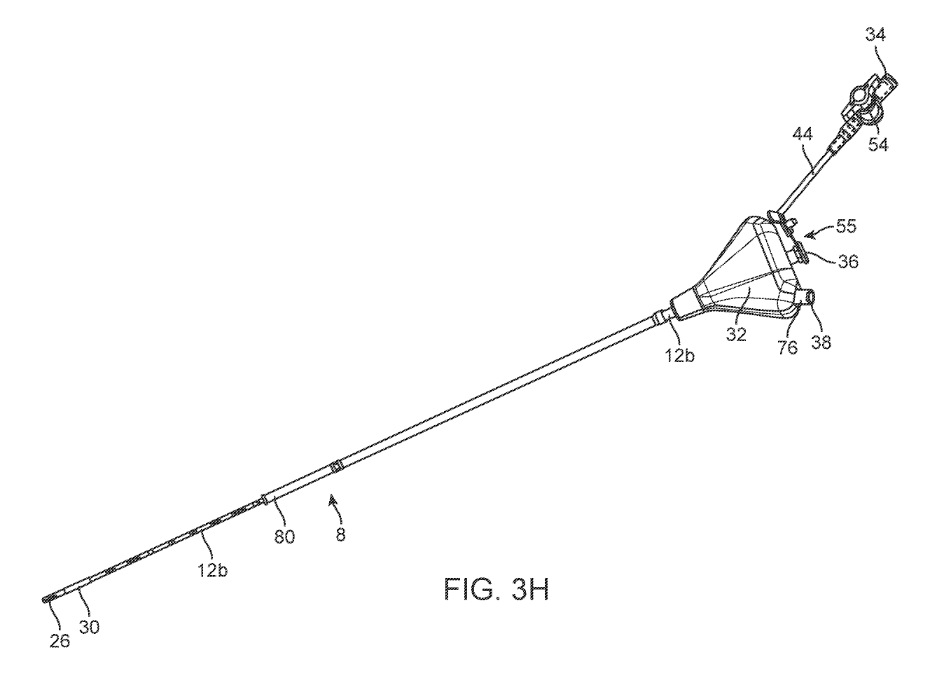

FIGS. 3a-3d illustrate a variation of an aeration system 10. As shown in FIG. 3a, the system 10 can have an inflation balloon 30, a dual lumen tubing 12 (e.g., first and second tubes 12a, 12b), and a connector 32. The inflation balloon 30 can be at a distal end of the system 10. The inflation balloon 30 can be inflated and deflated. The connector 32 can be a four-way connector. The connector 32 (e.g., four-way connector 32) can connect to or otherwise be in fluid communication with a fluid source for the inflation balloon 30, a fluid source for the first tube 12a, a fluid source for the second tube 12b, and an outlet port 26. The fluid sources for the inflation balloon 30, the first tube 12a, and the second tube 12b can be a gas and/or a liquid (e.g., gas 16 and/or liquid 18).

The system 10 can have one or more inlet ports and one or more outlet ports. For example, the system 10 can have an inlet port 34 for the balloon 30, an inlet port 36 for the first tube 12a, an inlet port 38 for the second tube 12b, and an outlet port 26. The outlet port 26 can be defined by at least a portion of the dual lumen tubing 12 (e.g., second tube 12b). The system 10 can have a tubing 44 that fluidly connects the inlet port 34 to the connector 32 and to the balloon 32. The system 10 can have a tubing 48 that fluidly connects the inlet port 38 to the connector 32 and to the second tube 12b of the dual lumen tubing 12. Although not shown in FIG. 3a, the first tube 12a can be an eductor tube within the second tube 12b. The proximal terminal end 15b of the second tube 12b can be within or at an entrance port of the connector 32, or anywhere along the length of the catheter 8.

The system 10 can have one or more flow control mechanisms. For example, the system 10 can have a mechanism 54 (e.g., a stopcock) between the inlet port 34 and the tubing 44 to control the flow of fluid into and out of the balloon 30. The system 10 can have a mechanism 58 (e.g., a stopcock) between the inlet port 38 and the tubing 48 to control the flow of fluid into the second tube 12b. The system 10 can have a mechanism 55 (e.g., a plug) in the inlet port 36 to control the flow of fluid into the first tube 12a. The plug 55 can be a gas plug. The plug 55 can be a liquid plug. Other flow control mechanisms are also appreciated.

FIG. 3b is a magnified view of the inflation balloon of FIG. 3a at section 3b-3b. The balloon 30 can be inflated and deflated. FIG. 3b shows the balloon 30 in an inflated configuration.

FIG. 3c is a transparent magnified view of the dual lumen tubing of FIG. 3a at section 3c-3c. FIG. 3c illustrates that the first tube 12a can be placed within the second tube 12b and/or the outlet channel 24 in a free-floating manner (e.g., a free-floating air lumen within the fluid lumen of the insertion catheter 8). Similarly, FIG. 3c illustrates that the central lumen 14a (not shown) can be placed within the second lumen 14b and/or the outlet channel 24 in a free-floating manner (e.g., a free-floating air lumen within the fluid lumen of the insertion catheter 8). The distal terminal end 13a of the first lumen 14a (e.g., air lumen) can be adjacent to the internal wall of the fluid lumen 14b and/or can be against the internal lumen of the fluid lumen 14b. For example, the distal terminal end 13a of the first lumen 14a can be adjacent an internal wall of the second tube 12b and/or can be against an internal wall of the second tube 12b. In a free-floating variation, the distal end of the first lumen 14a (e.g., air lumen) would tend to be off the central axis 28 since in a free-floating system there is not a mechanism to keep the distal end of the internal lumen away from the internal wall. This maybe particularly true for catheters that are inserted into the body. The various curves and tortuosity of insertion device within the body can stress the first lumen 14a (e.g., air lumen) laterally away from the central axis 28. The first lumen 14a (e.g., air lumen) can entrain air bubbles at a clinically acceptable level. The system 10 in FIGS. 1 and 2 can have free-floating configurations. For example, the first tube 12a in FIGS. 1 and 2 can be within the second tube 12b such that the distal terminal end 13a of the first tube 12a can freely float within the second tube 12b.

FIG. 3d is a transparent magnified view of the housing of FIG. 3a at section 3d-3d. FIG. 3d illustrates that the first tube 12a (e.g., eductor tube 12a) can be connected to a filter 72. The filter 72 can be in fluid communication with the inlet port 36 and the first tube 12a. The filter 72 can be between the first tube 12a and the inlet port 36. For example, the filter 72 can be between the proximal terminal end 15a of the first tube 12a and the inlet port 36. The filter 72 can be, for example, a 0.2 micron filter. However, any suitable filter is appreciated. FIG. 3d illustrates that the tube 44 can be in fluid communication with a connector tubing 74. The connector tubing 74 can be in fluid communication, directly or indirectly, with the balloon 30.

FIGS. 3e-3h illustrate a variation of an aeration system 10. FIG. 3e illustrates that the system 10 can have a valve 76, a strain relief 78, and a mandrel 80. The valve 76 can, for example, control the flow of fluid (e.g., gas 16 or liquid 18) into and/or out of the system 10. FIG. 3e illustrates that the distal tip of the catheter can be about 4 inches (e.g., 4.13 inches) from the mandrel 80. Other values, more or less, are also appreciated (e.g., less than 2 inches, less than 4 inches, less than 6 inches, or 6 inches or more). FIG. 3e illustrates that the distal tip of the catheter can be about 12 inches (e.g., 11.8 inches) from the connector 32. Other values, more or less, are also appreciated (e.g., less than 10 inches, less than 12 inches, less than 14 inches, or 14 inches or more).

FIGS. 3e and 3f illustrate that the outlet port 26 can be at least partially on a wall of the second tube 12b (e.g., on the side of the second tube 12b). As shown, the outlet port 26 can be at a distal end of the catheter 8. The outlet port 26 can define at least a portion of the distal terminal end 13b of the second tube 12b.

FIG. 3g illustrates that a spacer 82 can be on (e.g., around) the first tube 12a. The spacer 82 can help to stabilize the position of the first tube 12a within the housing. The spacer 82 can be on the first tube 12a, for example, between the proximal terminal end 15a of the first tube 12a and the proximal terminal end 15b of the second tube 12b.

FIG. 3h illustrates a perspective view of the system 10 of FIGS. 3e-3g. Various components are shown transparent for illustrative purposes.

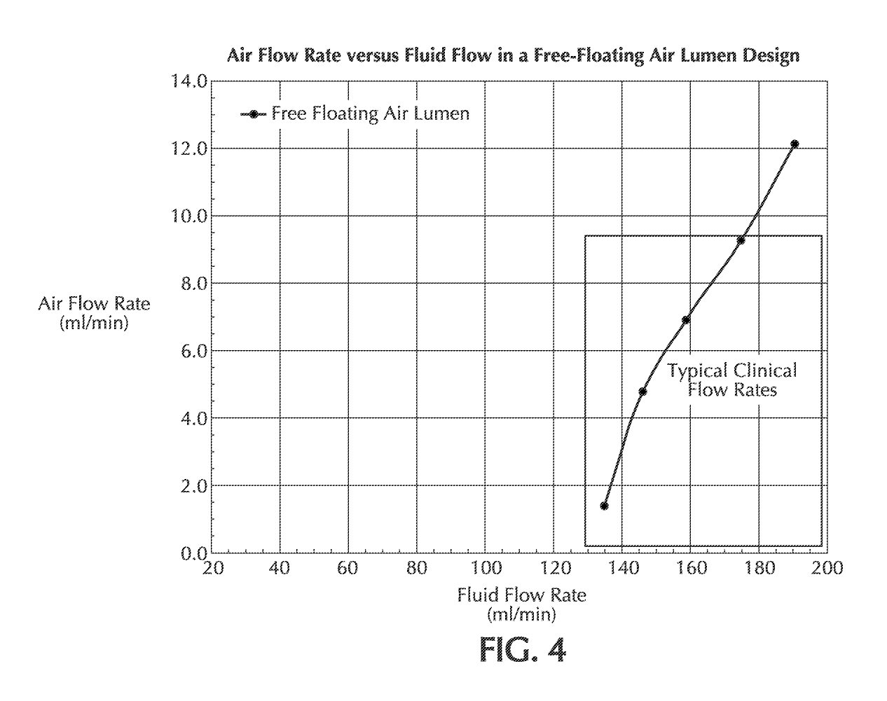

The free-floating configuration has been demonstrated to provide sufficient air bubble volumes with normal fluid flow rates. FIG. 4 is a graph showing the performance of an aeration system having a free-floating air lumen within a catheter (e.g., catheter 8) using various fluid flow rates.

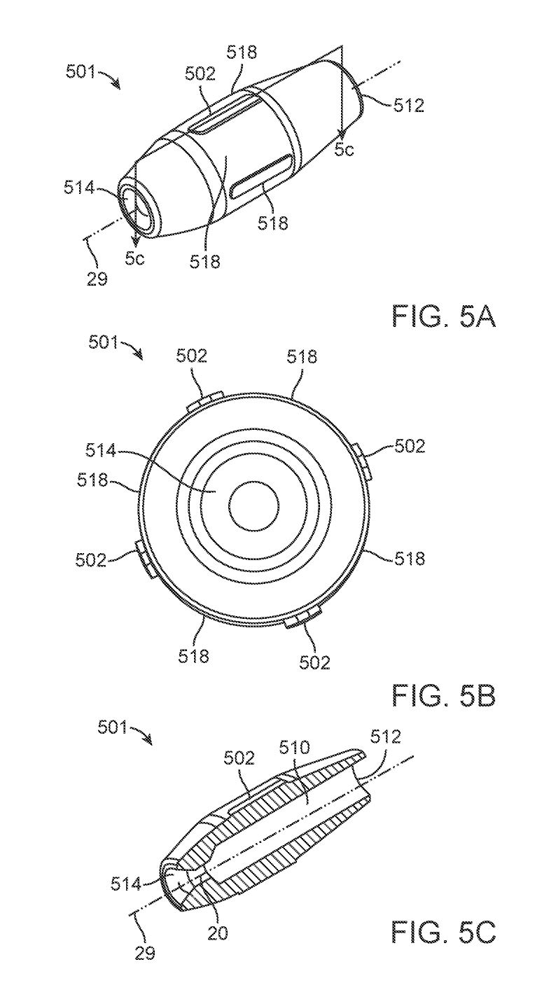

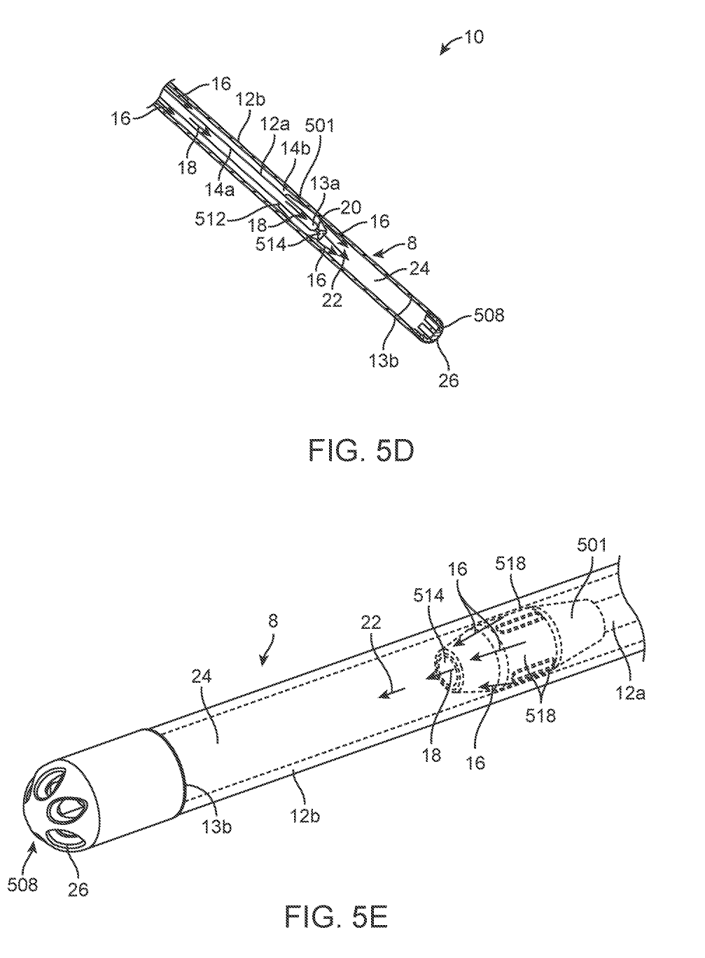

FIGS. 5a-5e illustrate that the aeration system 10 can have an inline eductor insert 501. FIG. 5a is a perspective view of a variation of the inline eductor insert 501. FIG. 5b is a front view of the eductor insert 501 of FIG. 5a. FIG. 5c is a longitudinal cross-sectional view of FIG. 5a take along line 5c-5c. FIG. 5d is longitudinal cross-sectional view of a variation of an aeration system 10 having the inline eductor insert 501 of FIGS. 5a-5c. FIG. 5e is a perspective view of the system 10 of FIG. 5d. The second tube 12b in FIG. 5e is shown transparent for purposes of illustration.

The inline eductor insert 501 can be close to and/or within the distal end of the catheter 8, including anywhere along the length of the catheter 8. The inline eductor insert 501 can be against a wall of the second tube 12b of an aeration system (e.g., system 10). For example, the inline eductor insert 501 can be pressed into the outer tube wall of the fluid tube 12b of the insertion catheter 8. The eductor insert 501 can be attached (e.g., welded) to the inner wall of the second tube 12b. The eductor insert 501 can have a lumen 510 and one or more ports. For example, the eductor insert 501 can have a first port 512 and a second port 514. The first port 512 can be a proximal port and the second port 514 can be a distal port. The inner lumen 510 in the eductor insert 501 can narrow into a throat 20 (also referred to as a venturi).

Fluid (e.g., gas 16, liquid 18) can flow through the lumen 510 of the insert 501. The lumen 510 can allow fluid (e.g., fluids 16, 18) to flow through the eductor insert 501. The inline eductor insert 501 can have one or multiple outer flow ridges 502 on an outer surface. The one or multiple flow ridges can allow fluid to flow outside of the insert 501. The one or multiple flow ridges 502 can allow fluid to flow past the insert 501 along an outer surface of the insert 501. The one or multiple flow ridges 502 can allow fluid to flow past the insert 501 within the second lumen 14b of the second tube 12b. The one or more ridges 502 can define one or more fluid channels 518 between the eductor insert 501 and a wall of the second tube 12b such that fluid can flow along the outside of the insert 501 from a first end to a second end.

FIG. 5b illustrates that the eductor insert 501 can have four ridges 502 and define four flow channels 518. As shown, each flow channel 518 can be defined between two ridges 502. Other numbers of ridges, more or less, are also appreciated (e.g., 10 or less, more than 10, among others). Other numbers of fluid channels 518, more or less, are also appreciated (e.g., 10 or less, more than 10, among others).

FIG. 5c illustrates that a length of the first tube 12a can be within the lumen 510 of the eductor insert 501. An end of the first tube 12a can be attached to or integrated with the eductor insert 501. For example, an end of the first tube 12a can be attached to the venturi 20 of the eductor insert 501. For example, a smaller air tube 12a can be bonded centrally into the proximal end of the insert 501 and/or to the venturi 20 of the insert 501. The insert 501 can have one or more internal venturi openings (not shown). Although only one venturi opening 20 is shown in FIGS. 5a-5e, the insert 501 can have multiple internal venturi openings 20. The one or multiple venturi openings can be along the length of the eductor insert 501, including at the proximal and/or distal ends. For example, the eductor insert 501 can have one or more distal venturi openings. The one or more internal venturi openings of the eductor insert 501 can increase aeration of the fluid (e.g., liquid 18, fluid 22).

The venturi 20 of the eductor insert 501 can be defined by the lumen 510. The lumen 510 can decrease (e.g., taper) from a first cross sectional area to a second cross sectional area. The lumen 510 can increase (e.g., taper) from the second cross sectional area to a third cross sectional area. The second cross-sectional area can be less than the first cross-sectional area and less than the third cross-sectional area. The first cross sectional area can be less than, equal to, or greater than the third cross sectional area. For example, a wall of the eductor insert 501 can change in diameter (e.g., internal diameter) at the throat 20. As shown in FIG. 5c, the wall of the eductor insert 501 can decrease from a first diameter to a second diameter (e.g., proximally to distally) and can increase from the second diameter to a third diameter (e.g., proximally to distally). The third diameter can be greater than, equal to, or less than the first diameter.

FIG. 5d illustrates that the inline eductor insert 501 can be within the second tube 12b. For example, the eductor insert 501 can be within the second lumen 14b and/or within the outlet channel 24. As shown, the eductor insert 501 can be inside the outlet channel 24 at the distal end 13a of the smaller air tube 12a within the insertion catheter 8. The inline eductor insert 501 can be coaxial with the insertion catheter 8 (e.g., with the second tube 12b). As described above, the first lumen 14a can be a gas lumen/conduit and the second lumen 14b can be a liquid lumen/conduit, or vice versa. Likewise, the lumen 510 of the eductor insert 501 can be a gas lumen/conduit and the one or more fluid channels 518 between the eductor insert 501 and the wall of the second tube 12b can be one or more liquid lumens/conduits, or vice versa. For example, the variation of the system 10 illustrated in FIG. 5d shows that the first lumen 14a and the lumen 510 of the eductor insert 501 can be conduits for the liquid 18, and that the second lumen 14b and the one or more channels 518 between the eductor insert 501 and the wall of the second tube 12b can be conduits for the gas 16. The gas 16 (e.g., air) can flow from a first part of the outer lumen 14b to a second part of the outer lumen 14b proximal to the insert 501, flow past the outer flow ridges 502 and through the one or more channels 518. and become entrained with the liquid 18 distal to the insert 501 to create an aerated liquid 22 flow distal to the insert 501, as shown by arrows. The liquid 18 can flow from a first part of the central lumen 14a to a second part of the central lumen 14a proximal to the insert 501, flow through the central lumen 510 and venturi 20 of the insert 501 (e.g., increasing in speed as the fluid flows through the venturi 20), and flow distal to the insert 501, mixing with the gas flow 16 (e.g., air flow) to become an aerated liquid 22 in the insertion catheter 8 distal to the insert 501.

FIG. 5d illustrates that the catheter 8 can have a catheter distal tip 508. The catheter distal tip 508 can have a rounded, atraumatic terminal surface. The catheter distal tip 508 can have one or more catheter outlet ports 26 (also referred to as distal ports). The catheter distal ports 26 can be located at the radial center of the terminal distal end of the tip 508, extending proximally along the sides of the tip, or combinations thereof. The catheter distal tip 508 can be attached to or integrated with the catheter 8. For example, the catheter distal tip 508 can be attached to or integrated with the second tube 12b (e.g., at the distal terminal end 13b of the second tube 12b).

FIG. 5e is a perspective view of the system 10 of FIG. 5d. The second tube 12b in FIG. 5e is shown transparent for purposes of illustration. FIG. 5e illustrates that the gas 16 can flow through the one or more channels 518 in the second tube 12b.

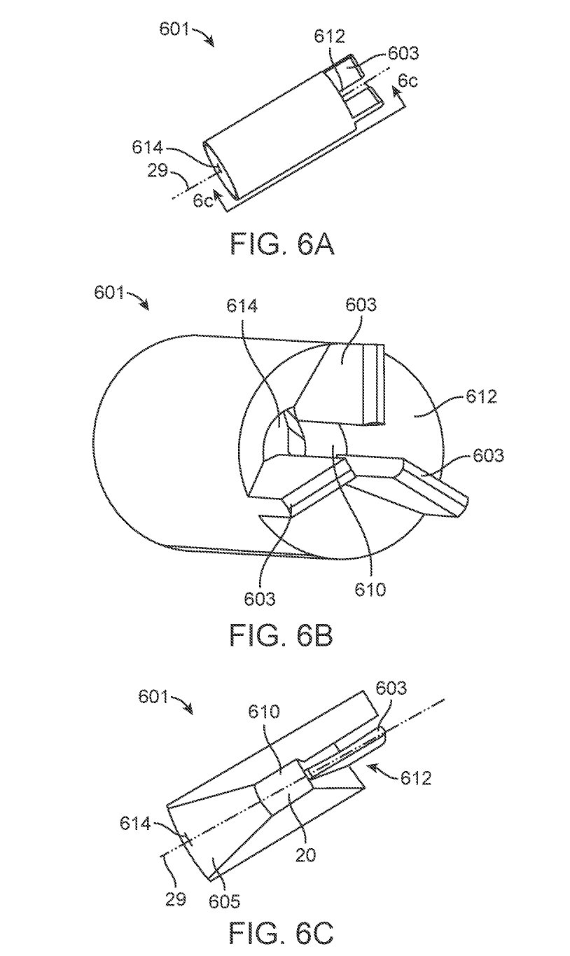

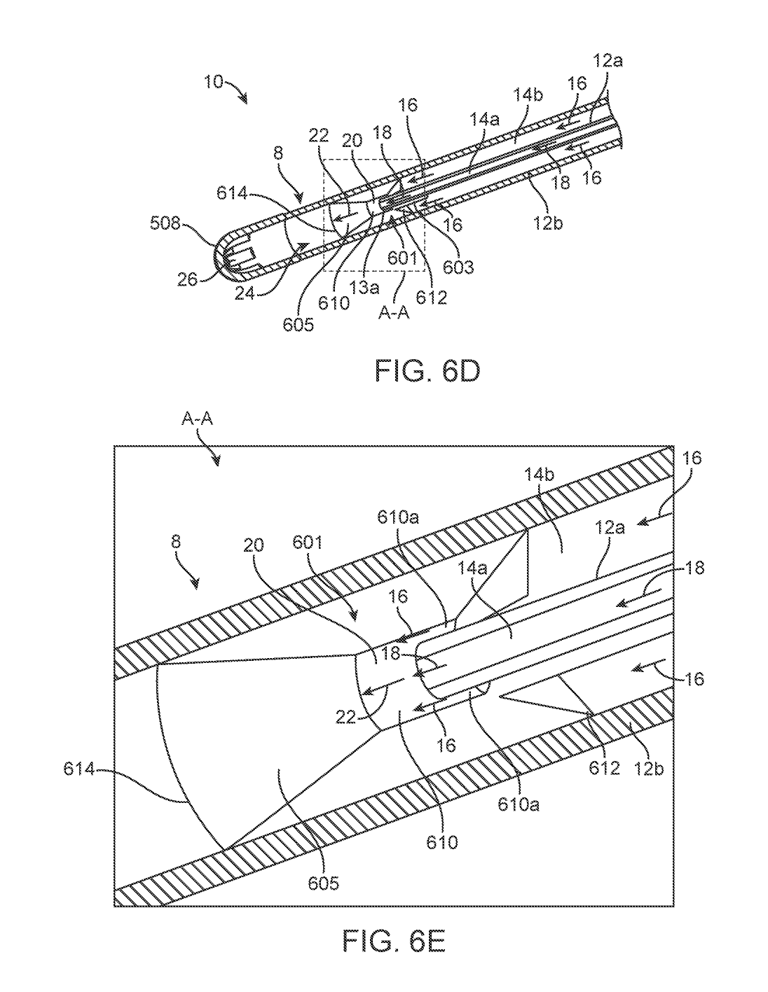

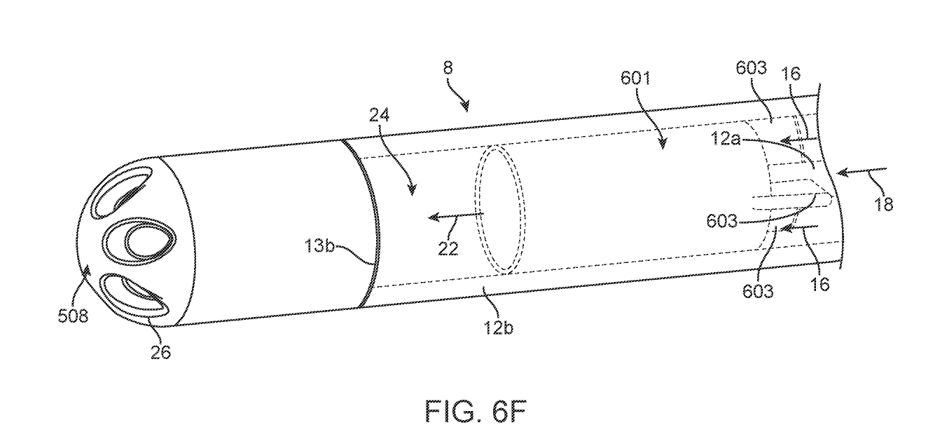

FIGS. 6a-6f illustrate that the aeration system 10 can have an inline eductor insert 601. FIG. 6a is a perspective view of a variation of the inline eductor insert 601. FIG. 6c is a longitudinal cross-sectional view of FIG. 6a take along line 6c-6c. FIG. 6d is longitudinal cross-sectional view of a variation of an aeration system 10 having the inline eductor insert 601 of FIGS. 6a-6c. FIG. 6e is a magnified view of section A-A of the variation of FIG. 6d. FIG. 6f is a perspective view of the aeration system of FIG. 6d.

The inline eductor insert 601 can be close to and/or within the distal end of the catheter 8, including anywhere along the length of the catheter 8. The inline eductor insert 601 can be against a wall of the second tube 12b of an aeration system (e.g., system 10). For example, the inline eductor insert 601 can be pressed into the outer tube wall of the fluid tube 12b of the insertion catheter 8. The eductor insert 601 can have a lumen 610 and one or more ports. For example, the eductor insert 601 can have a first port 612 and a second port 614. The first port 612 can be a proximal port and the second port 614 can be a distal port.

FIGS. 6a-6f illustrate that the eductor insert 601 can have one or more fins 603. The one or more fins 603 can each extend radially from an outer radius to an inner radius toward a longitudinal axis 29 of the eductor insert 601. The outer radii can be flush with an outer surface of the eductor insert 601. The one or more fins 603 can each extend proximally away from the distal port 614. The one or more fins 603 can direct fluid from the second lumen 14b into the lumen 610 of the eductor insert 601. The inner lumen 610 in the eductor insert 601 can narrow into a venturi 20. The one or more fins can form part of the venturi 20, narrowing the flow path of the second lumen 14b into the lumen 610 of the eductor insert 601.

The fluids 16, 18 can flow through the lumen 610 of the eductor insert 601. A length of the first tube 12a can be within the lumen 610 of the eductor insert 601. An end of the first tube 12a can be attached to or integrated with the eductor insert 601. For example, an end of the first tube 12a can be attached to the eductor insert 601. For example, a smaller air tube 12a can be bonded to the one or more proximal fins 603 of the insert 601. The smaller air tube 12a can be bonded centrally to the one or more proximal fins 603 of the insert 601. The fluid can flow into the proximal end of the insert 601 outside of the inner air tube 12a. The insert 601 can have one or more internal venturi openings (not shown). Although only one venturi opening 20 is shown in FIGS. 6a-6f, the insert 501 can have multiple internal venturi openings 20. The one or more internal venturi openings can be along the length of the eductor insert 601, including at the proximal and/or distal ends. For example, the eductor insert 601 can have one or more distal venturi openings. The one or more internal venturi openings of the eductor insert 601 can increase aeration of the fluid (e.g., liquid 18, fluid 22).

FIG. 6b illustrates that the eductor insert can have three fins. The three fins can define a space for receiving the first tube 12a. As described above, the first tube 12a can be attached to the fins 603. The fins 603 can maintain the distal terminal end 13a of the first tube 13a within the lumen 610 (see e.g., FIG. 6e). The fins 603 can maintain the distal terminal end 13a of the first tube 13a within the lumen 610 in a constant radial dimension away from the wall of the lumen 610. Other numbers of fins, more or less are also appreciated (e.g., 10 fins or less, greater than 10 fins).

FIGS. 6b-6d illustrate that the eductor insert 601 can have a nozzle 605. The nozzle 605 can be at the distal end of the eductor insert 601. The nozzle 605 can facilitate the mixing of the gas 16 and the liquid 18.

FIG. 6d illustrates that the inline eductor insert 601 can be within the second tube 12b similar to how the eductor insert 501 is within the second tube 12b (see e.g., FIG. 5c).

FIG. 6e is a magnified view of section A-A of the variation of FIG. 6d. As described above, the first lumen 14a can be a gas lumen/conduit and the second lumen 14b can be a liquid lumen/conduit, or vice versa. At least a portion of the lumen 610 of the eductor insert 601 can be a gas conduit and/or a liquid conduit. For example, the variation of the system 10 illustrated in FIG. 6d shows that the first lumen 14a can be a conduit for the liquid 18, and that the second lumen 14b and at least a first portion 610a of the lumen 610 of the eductor 601 can be conduits for the gas 16. The gas 16 (e.g., air) can flow from a first part of the outer lumen 14b to a second part of the outer lumen 14b proximal to the insert 601, flow past the one or more fins 603 and into the first portion 610a of the lumen 610 (e.g., increasing in speed as the fluid flows past the fins 603), and become entrained with the liquid 18 at a position distal to the first portion 610a of the lumen 610 to create an aerated liquid 22 flow distal to the insert 601, as shown by arrows. For example, the gas 16 can begin to become entrained with the liquid 18 in the second portion 610b of the lumen 610. The liquid 18 can flow from a first part of the central lumen 14a to a second part of the central lumen 14a proximal to the insert 601, flow past the first portion 610a of the lumen 610 (e.g., while within the first tube 12a), and begin mixing with the gas flow 16 (e.g., air flow) to become an aerated liquid 22 in the second portion 610b of the lumen 610. The distal nozzle 605 can further aerate the gas and liquid 16, 18 by creating turbulence in the flow stream. This can advantageously decrease the size of the bubbles that make up the aerated liquid 22.

FIG. 6f is a perspective view of the system 10 of FIGS. 6d and 6e. The second tube 12b in FIG. 6f is shown transparent for purposes of illustration. FIG. 6f illustrates that the eductor insert 601 can be placed near the catheter distal tip 508.

Although not shown in FIGS. 6a-6f, the eductor insert 601 can have the one or more ridges 502 and/or the one or more fluid channels 518 described above with reference to eductor insert 501.

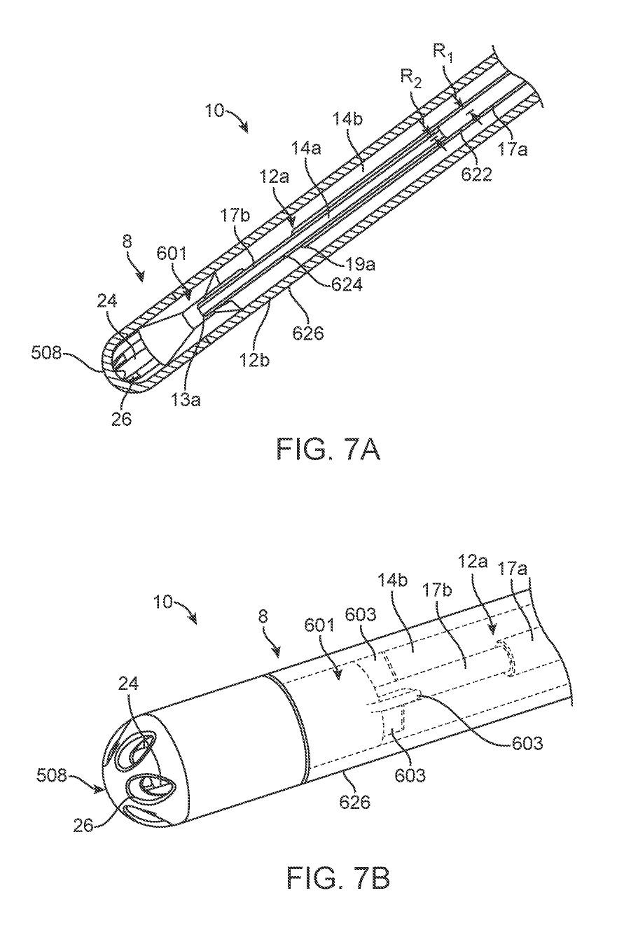

FIG. 7a illustrates that the inline eductor insert 601 can be close to and/or within the distal end of the catheter 8, for example within the catheter distal tip 508. As shown, the eductor insert 601 can be within the most distal end of the catheter 8. The inner (e.g., liquid or gas) tube 12a can be reduced in diameter to make smaller diameter bubbles. The inner tube 12a can have an inner tube proximal wall 622 and an inner tube distal wall 624. The inner tube 12a can comprise a first inner tube 17a and a second inner tube 17b. The first and second inner tubes 17a, 17b can define the walls 622, 624, respectively. The radially inner side of the distal end of the inner tube proximal wall 622 can have an air-tight bond (e.g., weld, epoxy) to the radially outer side of the proximal end of the inner tube proximal wall 624. The inner radius R₁ of the inner tube proximal wall 622 can be larger than the inner radius R₂ of the inner tube distal wall 624. The inner radius R₁ of the first inner tube 17a can be larger than the inner radius R₂ of the second inner tube 17b. The inner radius R₁ can range from 0.01 inches to 0.1 inches. Other ranges for the inner radius R₁, narrower or wider, are also appreciated. The inner radius R₂ can range from 0.005 inches to 0.05 inches. Other ranges for the inner radius R₂, narrower or wider, are also appreciated. The distal terminal end 13a of the second inner tube 17b can be closer to the eductor insert 601 and/or the outlet port 26 than the distal terminal end 19a of the first inner tube 17a.

FIG. 7b is a perspective view of the system 10 of FIG. 7a.

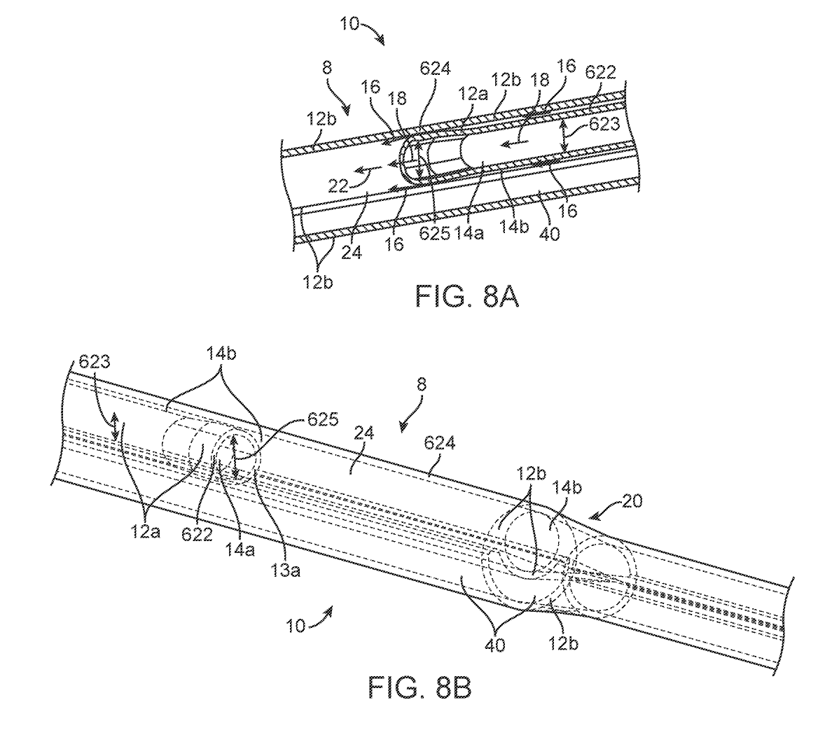

FIG. 8a illustrates that the inner (e.g., liquid or gas) tube 12a can be distally flared, for example expanded and shaped distally to form an eductor shape similar in shape to the inline eductor inserts described above. The distally flared air tube can be used in an aerator system 10 with or without an eductor insert. The expanded air tube (e.g., first tube 12a) can be within the second tube 12b and/or within the distal catheter tip 508.

The proximal end of the inner tube 622 can have a proximal inner tube wall diameter 623. The distal end of the inner tube 12a can have a distal inner tube wall inner diameter 625. The proximal inner tube wall diameter 623 can be less than the distal inner tube wall inner diameter 625.

FIG. 8a illustrates that the catheter 8 can have one or more lateral lumens 40. The one or more lateral lumens 40 can be on a lateral side of the outer lumen 14b of the catheter 8, and/or can be one or more supplemental external coaxial lumens outside of the outer tube 12b (e.g., outside of a wall of the outer tube 12b). One or more tubes (e.g., tubes 12a, 12b) can form the one or more lateral lumens 40. For example, the second tube can form the second lumen 14b and/or one or more of the one or more lateral lumens 40. Additional gasses, liquids, instruments or tools, deflecting mandrels for distal end articulation, stiffening mandrels to increase catheter stiffness, or combinations thereof can be inserted into and/or through the one or more lateral lumens 40 and/or one or more supplemental external lumens.

The expanded or flared air inner tube 12a can have one or more splines (not shown) on the internal and/or external surfaces of the inner tube wall, traversing the inner tube wall, and/or in, on, and/or traversing the outer tube wall near the distal end, for example within the central (e.g., inner) and/or outer lumens. The splines can brace the inner tube at a constant distance along the length of the inner tube from the inner surface of the outer tube wall.

The splines can have bumps and ridges on the distal end of the inner (e.g., liquid or gas) lumen 14a, for example to create spacing for fluid flow and creating the venturi effect. The inner tube 12a can be made from stainless steel tubing and/or a thermoplastic formed, drawn, or extruded into a tube. At the distal end of the air inner tube 12a, a crimping tool can be used to create ridges and bumps on the terminal distal end to shape the tube, for example to change air or liquid flow during use.

The crimping tool can be used to crimp the outer (e.g., fluid) tube 12b to create ridges and/or bumps to change fluid flow, as described above for the air tube.

FIG. 8b is a perspective view of the system 10 of FIG. 8a. The catheter 8 is shown transparent for purposes of illustration. FIG. 8b illustrates that the second tube 12b can form the second lumen 14b and the one or more lateral lumens 40. As shown, the second lumen 14b can have a circular cross-section and the lateral lumen 40 can have a crescent-shaped cross-section. However the second and lateral lumens 14b, 40 can have any shaped cross-section, including circular, square, polygonal, curved and/or angular.

FIG. 8b illustrates that the second tube 12b can form a venturi 20. The venturi 20 can be formed like the venturi 20 described above with reference to FIG. 2. The venturi 20 can further aerate the fluid 22, for example, to make smaller diameter bubbles or microbubbles for enhanced echogenicity.

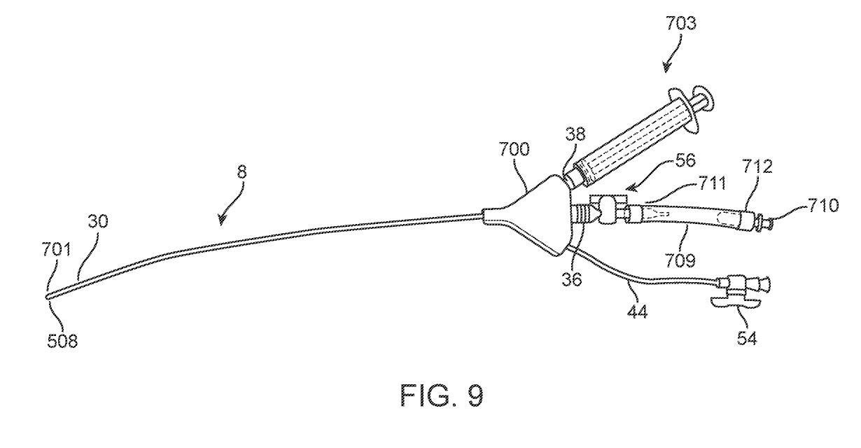

FIG. 9 illustrates that the catheter 8 can have a proximal handle 700 and a vessel 709. The handle 700 can include the connector 32 described above. The proximal handle 700 can have a fluid source 703 attached to a fluid (e.g., liquid) injection port 38. The fluid source 703 can be a syringe (e.g., a syringe filled with saline), a pressurized fluid source, a gravity fed fluid source, a fluid pump, a syringe pump, a gear pump, or a stepper motor, each of which can be designed to provide fluid (e.g., non-aerated liquid) into the fluid injection port 702 and into catheter 8.

The proximal handle 700 can have a balloon inflation conduit 44 with a stopcock 54 to control the inflation and deflation of an anchoring balloon 30 on a distal end 701 of catheter 8. The balloon 30 can anchor the tip 508 of the catheter 8 relative to the uterus and/or fallopian tube and/or peritoneal cavity.

The proximal handle 700 can have a fluid port 36 (e.g., gas port or liquid port) connected to the inner (e.g., air or liquid) lumen 12a within the catheter 8 and the eductor insert, venturi, throat, or restriction (see e.g., eductor insert, venturi, throat, or restriction 501 or 601). The gas port 36 (e.g., air port) can be connected to an air filter as a sterile air barrier (not shown). The fluid port 36 can be connected to a stopcock 56. The fluid port 36 can be connected to the vessel 709.

The system 10 can have one or more vessels 709. The vessel (e.g., vessel 709) can hold a volume of fluid. For example, the vessel 709 can hold a volume of gas (e.g., air) and/or liquid. The vessel 709 can have any suitable volume capacity. For example, the vessel 709 can have a capacity of 5 cc, 10 cc, or 15 cc. Other volume capacities, more or less, are also appreciated (e.g., less than 5 cc, less than 10 cc, less than 15 cc, less than 20 cc, more than 15 cc, among others). The vessel 709 can be inflated and deflated. The vessel 709 can be partially and/or fully inflated and deflated. For example, a vessel 709 with a 10 cc capacity can be filled with 10 cc or less of fluid and the 10 cc or less of fluid can be deflated from the vessel 709 in one or more increments.

The stopcock 56 can be used to control the flow of fluid into the catheter 8 (e.g., into the first tube 12a) from the vessel 709. The vessel 709 can have a valve 710. The valve 710 can be a luer activated check valve, a one-way valve, a stopcock (e.g., stopcock 54, 56, 58, among others), or other open/close valve apparatuses. The valve 710 can be normally open or normally closed. The vessel 709 can be attached to the stopcock 56 with a first connector 711 (e.g., a distal connector). The valve 710 can be attached to the vessel 709 with a second connector 712 (e.g., a proximal connector). The stopcock 56 and the valve 710 can be attached to the vessel 709 by bonding, welding, or other catheter assembly techniques. The vessel 709 can supply/deliver gas (e.g., air) bubbles on demand and work in conjunction with eductor/aspirator for creation/formation of micro-bubbles.

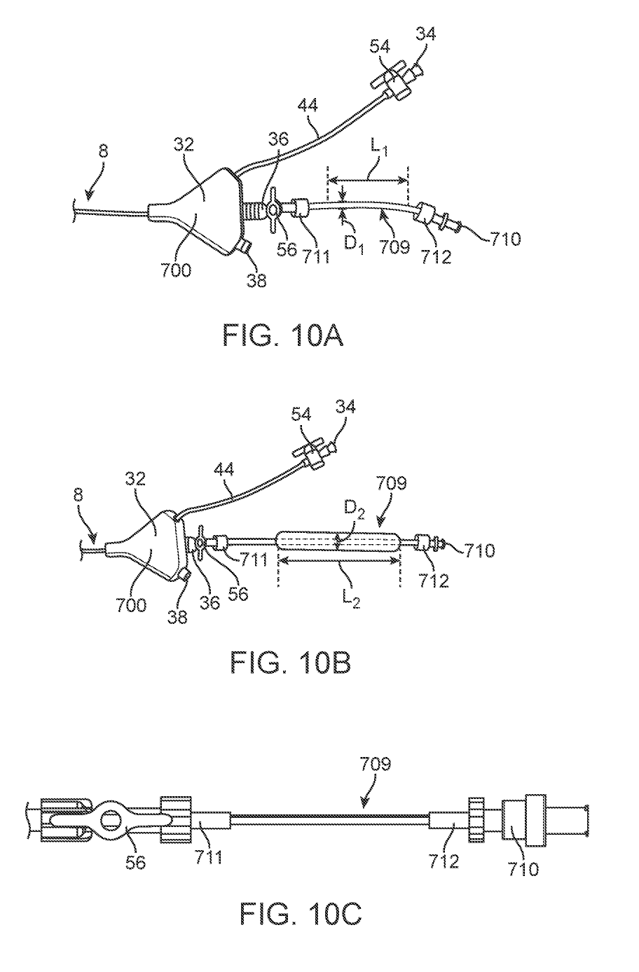

FIG. 10a illustrates the vessel 709 in an unexpanded (e.g., deflated) configuration. FIG. 10b illustrates the vessel 709 in an expanded (e.g., inflated) configuration. FIG. 10c illustrates the vessel 709 of FIGS. 10a and 10b in an unexpanded configuration. The vessel can be non-pressurized and/or pressurized relative to a reference pressure (e.g., atmospheric pressure). For example, the pressure in the vessel can be equal to, below (e.g., negative), or above (e.g., positive) relative to atmospheric pressure. The vessel 709 can hold non-pressurized and/or pressurized fluid (i.e., the vessel 709 can be in a non-pressurized state, a negative pressure state, and/or a positive pressure state relative to atmospheric pressure when in an expanded configuration). For example, the vessel 709 can have a pressure equal to, below, and/or above atmospheric pressure when in an expanded configuration shown in FIG. 10b. The vessel can hold the gas 16, the fluid 18, and/or the aerated fluid 22.

FIGS. 10a and 10b illustrate that a diameter (or other dimension, e.g., length, width, height, radius, etc.) of the vessel 709 can be larger in the expanded configuration than in the unexpanded configuration. For example, FIG. 10a illustrates that the vessel 709 can have an unexpanded diameter D₁ and FIG. 10b illustrates that the vessel 709 can have an expanded diameter D₂. The unexpanded diameter (e.g., when fully deflated) D₁ can range from 0.05 inches to 0.5 inches. Other ranges for the unexpanded diameter D₁, narrower or wider, are also appreciated. The expanded diameter (e.g., when fully inflated) D₂ can range from 0.1 inches to 1.0 inches. Other ranges for the expanded diameter D₂, narrower or wider, are also appreciated.

FIGS. 10a and 10b illustrate that the vessel 709 can have a length L₁ in the unexpanded configuration and a length L₂ in the expanded configuration. The lengths L₁ and L₂ can have the same or substantially the same dimension (e.g., as shown in FIGS. 10a and 10b). The lengths L₁ and L₂ can be different from one another (e.g., the length of the vessel 709 can lengthen and/or shorten when inflated and/or deflated). The length L₁ of the vessel 709 in the unexpanded configuration (e.g., when fully deflated) can range from 1.0 inches to 10.0 inches. Other ranges for the length L₁, narrower or wider, are also appreciated. The length L₂ of the vessel 709 in the expanded configuration (e.g., when fully inflated) can range from 1.5 inches to 20.0 inches. Other ranges for the length L₂, narrower or wider, are also appreciated.

The vessel 709 can deliver fluid to a biological target site (e.g., via the catheter 8) and/or withdraw fluid from a biological target site (e.g., via the catheter 8). The vessel 709 can deliver fluid to the catheter 8 and/or withdraw fluid from the catheter 8. The vessel 709 can supply gas (e.g., gas 16) to the aerator system 10 to create air bubbles for echogenic contrast media in target sites. For example, the vessel 709 can supply gas at a positive pressure to the aerator system 10. The positive pressure can facilitate the formation of bubbles in the aerated fluid 22, for example, by increasing the venturi effect of the system 10. A vacuum can be created in the vessel 709. The vessel 709 can withdraw fluid (e.g., gas 16, liquid 18, and/or aerated fluid 22) from the target sites by exposing the target sites to the vacuum or negative pressure in the vessel 709 (e.g., via the one or more tubes or other features of the catheter 8 or via another separate device). The vessel 709 can thereby decrease the distension of the target sites when negative pressure is applied, making the ultrasound procedure more comfortable to the patient by preventing the target site from becoming overly or uncomfortably distended. In this way, the vessel 709 can apply suction to the system 10, the catheter 8, the tip 508 of the catheter 8, and/or the target site.

In operation, the physician or operator can inflate the vessel 709 with gas (e.g., air) using a syringe or other inflation device.

As described above, in use the physician or operator can insert at least a portion of the catheter 8 into a patient's body cavity (e.g., uterus, fallopian tubes and/or peritoneal cavity). The operator can use the anchoring balloon 30 to seal the body cavity in which the catheter 8 is inserted. The fluid source 703 (e.g., the syringe 703 shown in FIG. 9) can be used to inject fluid (e.g., saline) within the uterine cavity to perform sonohysterography or saline infused sonohysterography (SIS). For example, to assess tubal patency for an infertility evaluation of a female patient, the operator/physician can inject fluid from the fluid source 703 (e.g., syringe 703) into the uterine cavity of a patient to distend the uterine cavity and provide intrauterine pressure to allow fluid to flow through the fallopian tubes (i.e., The pressure in the uterine cavity can be increased by injecting fluid from the fluid source 703 into the uterine cavity of the patient. Once the intrauterine pressure is sufficiently increased, the injected fluid can flow through the fallopian tubes). The threshold intra-cavity (e.g., intrauterine) pressure in the uterine cavity that is required before the fluid will flow through the fallopian tubes is on average about 70 mmHg (including exactly 70 mmHg). For female patients with blocked fallopian tubes, the intra-cavity (e.g., intrauterine) pressure will not be sufficient to open or demonstrate open fallopian tubes. For example, the fallopian tubes may not open even when the pressure in intrauterine cavity is increased to 70 mmHg or more.

To facilitate ultrasound imaging of fallopian tube patency, gas (e.g., air) bubbles can be injected into the uterine cavity with the concurrent flow of liquid via injection by the syringe 703. For example, an air-saline contrast fluid can be injected into the uterine cavity in a procedure called sonohysterosalpingography. The air-saline contrast fluid can provide greater echnogenicity in comparison to other contrast fluids. In the echogenic catheter system variation illustrated in FIGS. 9-10B, the inflated pressurized vessel 709 can be opened with stopcock 56 to allow the flow of gas (e.g., air) into the lumen (e.g., first lumen 14a) of catheter 8. When the stopcock 56 is in an open configuration, gas (e.g., air) bubbles will exit the distal end 701 of catheter 8 into the distended uterine cavity and ultimately flow through the fallopian tubes where the echogenic air bubbles can be more easily seen by ultrasound visualization (if the fallopian tubes are sufficiently patent). In practice, these echogenic gas (e.g., air) bubbles can be further enhanced by the entrainment of the gas into the fluid flow when the fluid is injected by the fluid source (e.g., the syringe 703) into the catheter 8, and can be further enhanced by the venturi effect that the aeration system 10 provides.

The gas (e.g., air) bubbles can be injected into the uterine cavity without the concurrent flow of liquid via injection by the syringe 703. This can be particularly beneficial for the comfort of patients with distended uteri. In this situation, the physician/operator can maintain the ability to provide, for example, an air-saline contrast with compressible air bubbles without the requirement of simultaneous injection of fluid which is incompressible. As such, the physician/operator can gain additional visualization time for ultrasound without adding to patient discomfort.

The concurrent injection of gas from the gas source (e.g., vessel 709) and fluid from the fluid source 703 (e.g., syringe 703) into the catheter 8 and body cavity can advantageously supply an aerated liquid to the target site that has a greater volume of gas and/or that has gas bubbles that are of a smaller diameter (e.g., that are microbubbles). The increased gas volume and/or smaller bubbles can provide greater echnogenicity as compared to the echnogenicity when the injection of the gas and liquid is not concurrent.

The control of the supply of gas (e.g., air) bubbles can be controlled/manipulated with the stopcock 56 and/or one or more restrictors in the lumen, for example, the first and/or second lumens 14a, 14b. The one or more restrictors can be manufactured by reducing the internal diameter of the lumen (e.g., the first and/or second lumens 14a, 14b) and/or by inserting smaller diameter tubing or orifices. The restrictors can reduce the gas (e.g., air) flow rate from the vessel 709. The restrictors can be a valve mechanism that can modulate/adjust the flow rate.

One or more of the one or more restrictors can be located in the distal end 701 of catheter 8, in the air stopcock 56, or at any point within the gas (e.g., air) lumen.

The vessel 709 can supply gas (e.g., air) at a pressure within the range from 70 mmHg to 200 mmHg, or within the range from 70 mmHg to 150 mmHg. Other pressure values, more or less, as well as other ranges, narrower or wider are also appreciated (depending, for example, upon the body cavity or if higher pressures are required). Pressures greater than 70 mmHg are designed to overcome intracavitary pressures evident in distended uteri.

The vessel 709 can supply gas (e.g., air) flow at a positive pressure due to the resiliency of the elastic walls of the vessel 709 responding to the injection of the gas by the physician or operator. The vessel 709 can operate with a secondary or external force acting on the vessel 709. Other pressurized air mechanisms on the vessel 709 can include mechanically squeezing plates, manual plates or springs, air pumps, air canisters, or inflation sources with regulators. All of these mechanisms can be placed within the proximal handle 700. The gas (e.g., air) stopcock 56 can be connected directly to a CO₂ source that can be used in place of room air.

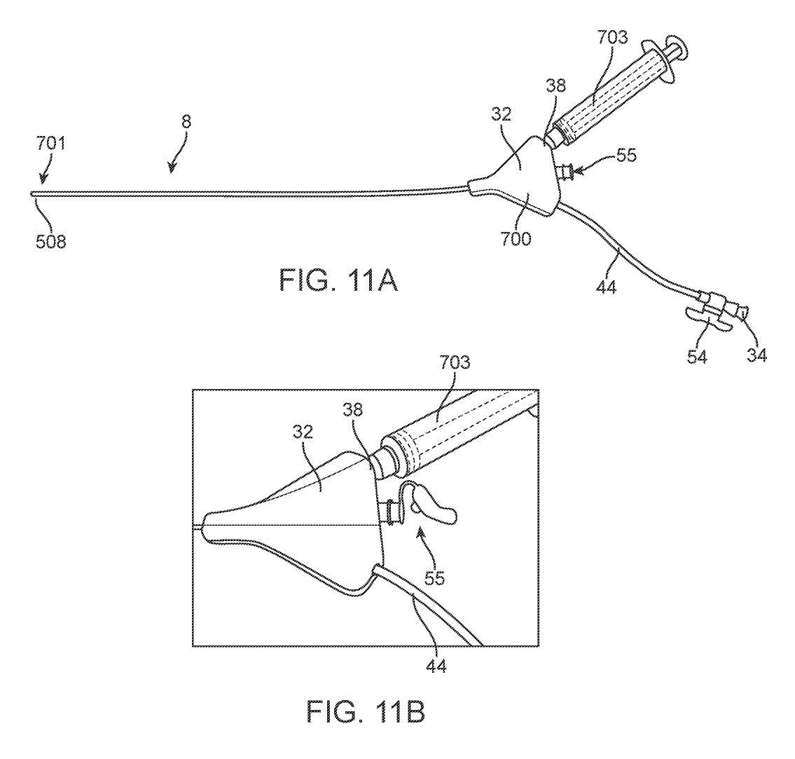

FIGS. 11a and 11b are similar to FIGS. 9-10b except that the aeration system 10 has a plug 55 instead of a stopcock 56. The plug 55 can be a gas and/or a liquid plug. FIG. 11a illustrates the plug 55 in a closed configuration and FIG. 11b illustrates the plug in an opened configuration. As shown in FIG. 11b, the plug 55 can have a removable cap attached to a body via a tether. A vessel 709 can be attached to the port 36 when the plug 55 is open. A fluid source 703 can be connected to the injection port 38 as shown in FIGS. 9-10b.

Internal ribs and spacers can be on the inner surface of the outer tube 12b of catheter 8 and/or on the outer surface of the central (i.e., inner) inner tube 12a, for example, protruding into the fluid lumen increasing fluid velocity and decreasing fluid pressure distally creating a venturi effect.

The aerator systems 10 can produce a venturi effect within the catheter 8 that does not require two co-linear catheter lumens for supplying fluid and air within an echogenic contrast media. The aerator systems 10 can supply sufficient air bubbles for echogenic contrast media in target sites.

The aerator systems 10 can be used to deliver aerated liquid to biological target sites, for example for echogenic contrast for visualization. For example, the aerator system can be used to deliver aerated saline solution to a uterus and/or fallopian tubes to visualize patency of fallopian tubes during ultrasound visualization. The target site can be the uterus, fallopian tubes, peritoneal cavity, or combinations thereof.

The aerator systems 10 can be used to deliver drugs, therapeutic agents, or biological material such as reproductive materials, into the uterus and/or fallopian tube and/or peritoneal cavity.

The aeration systems 10 can be used for the delivery of distension media, including CO₂ into the peritoneal cavity.

As used herein “air” can be air, carbon dioxide, nitrogen, oxygen, steam (water vapor), or combinations thereof. “Fluid” can be a liquid or gas, for example saline solution, water, steam, or combinations thereof.

The gas can be delivered through the inner or central lumen (e.g., lumen 14a) and the fluid can be delivered through the outer lumen (e.g., lumen 14b). The gas can be delivered through the outer lumen (e.g., lumen 14b) and the fluid can be delivered through the inner or central lumen (e.g., lumen 14a).

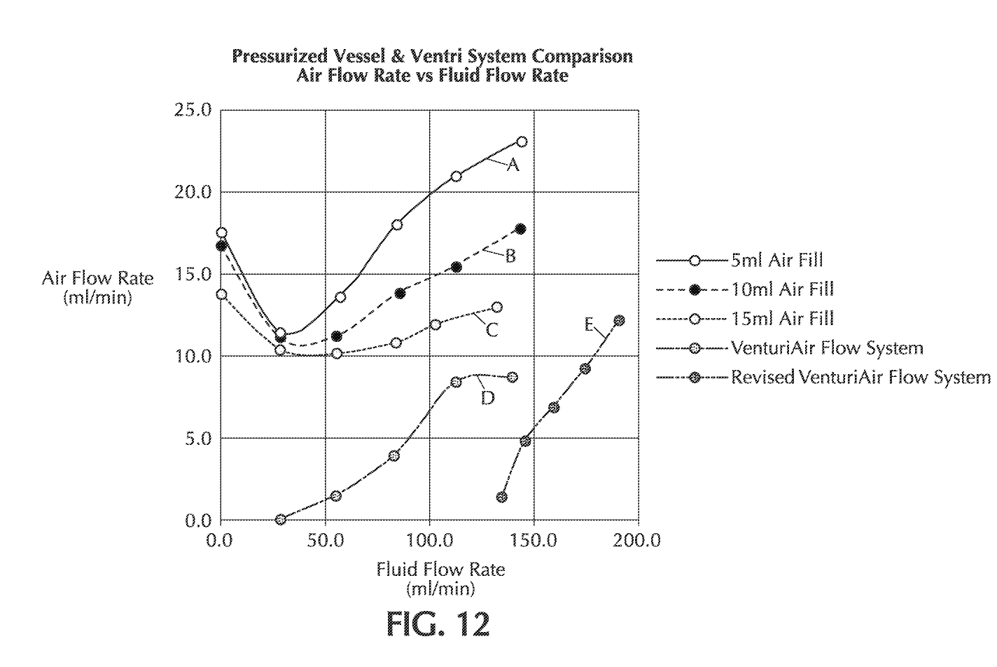

FIG. 12 is a graph illustrating air flow versus fluid flow for various aeration systems. The graph in FIG. 12 compares various pressurized vessel and venturi systems. Line A is for a system 10 having a 5 mL air fill. Line B is for a system 10 having a 10 mL air fill. Line C is for a system 10 having a 15 mL air fill. Line D is for a venturi air flow system 10 (e.g., FIGS. 1-8). Line E is for a venturi air flow system 10 (e.g., FIGS. 9-10b).

U.S. patent application Ser. No. 14/495,726, filed Sep. 14, 2014, U.S. Provisional Application Nos. 61/005,355, filed May 30, 2013; 61/977,478, filed Apr. 9, 2014; 62,007,339, filed June 3, 2014; and 61/902,742, filed Nov. 11, 2013, are each herein incorporated by reference in their entireties.

Any elements described herein as singular can be pluralized (i.e., anything described as “one” can be more than one). Like reference numerals in the drawings indicate identical or functionally similar features/elements. Any species element of a genus element can have the characteristics or elements of any other species element of that genus. “Dilation” and “dilatation” are used interchangeably herein. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

We claim:

1. An aerator system for use in a biological target site comprising:
an inner tube and an outer tube, wherein at least a portion of the outer tube overlaps the inner tube; and
a venturi element within the outer tube, wherein at least a portion of the venturi element extends beyond a distal end of the inner tube.

2. The system of claim 1, wherein at least a portion of the inner and outer tubes are coaxial with one another.

3. The system of claim 1, wherein the inner tube defines an inner lumen and the outer tube defines an outer lumen, wherein the inner tube lumen is configured to receive a gas, and wherein the outer tube lumen is configured to receive a liquid.

4. The system of claim 3, wherein the gas is air and the liquid is saline.

5. The system of claim 3, wherein the outer tube defines an outer lumen, wherein at least a portion of the outer lumen is around the inner tube, wherein at least a portion of the outer lumen is around the venturi element, and wherein at least a portion of the outer lumen extends beyond a distal end of the venturi element.

6. The system of claim 1, wherein an external surface of the venturi element comprises a flow channel.

7. The system of claim 6, wherein the flow channel is configured to increase the velocity of the liquid in the outer lumen as it flows past the venturi element.

8. The system of claim 1, wherein the venturi element comprises an eductor insert, and wherein the eductor insert is within the outer tube.

9. The system of claim 8, wherein an external surface of the eductor insert is attached to an inner surface of the outer tube.

10. The system of claim 1, wherein the outer tube defines an outer lumen, wherein at least a portion of the outer lumen is between the inner tube and the outer tube, and wherein the venturi element is configured to direct flow from the outer lumen radially outside the venturi element.

11. The system of claim 1, wherein the outer tube defines an outer lumen, wherein at least a portion of the outer lumen is between the inner tube and the outer tube, and wherein the venturi element is configured to direct flow from the outer lumen radially inside the venturi element.

12. The system of claim 1, wherein the outer tube defines an outer lumen, wherein at least a portion of the outer lumen is between the inner tube and the outer tube, and wherein the venturi element comprises a fin extending toward a proximal end of the outer lumen.

13. A method of delivering aerated liquid to a biological target site comprising:
inserting an aerator system into the target site, wherein the aerator system comprises an inner tube having an inner lumen, an outer tube having an outer lumen, and a venturi, wherein at least a portion of the inner and outer tubes are coaxial with one another, and wherein at least a portion of the outer lumen is between the inner tube and the outer tube;
delivering a liquid through the outer lumen;
aerating the liquid, wherein aerating comprises delivering a gas through the inner lumen; and
delivering the aerated liquid to the biological target site.

14. The method of claim 13, wherein aerating comprises concurrently delivering the gas through the inner lumen and delivering the liquid through the outer lumen.

15. The method of claim 13, wherein the biological target site comprises a uterus.

16. The method of claim 13, further comprising echogenically visualizing the biological target site.

17. The method of claim 13, wherein the venturi comprises an eductor insert within the outer tube.

18. The method of claim 13, wherein the venturi comprises a flared configuration of the distal end of the inner tube.

19. The method of claim 13, wherein the venturi comprises a radial narrowing of the inner surface of the outer tube from a first end toward a second end of the outer tube.

20. A method for using an aerator system in a biological target site, wherein the aerator system comprises an inner tube and an outer tube coaxial with the inner tube, wherein at least a portion of the outer tube overlaps the inner tube, the method comprising:
inserting a distal end of the outer tube into the biological target site;
delivering a liquid through the outer tube;
aerating the liquid, wherein aerating comprises delivering a gas through an inner tube; and
delivering the aerated liquid to the biological target site.

21. The method of claim 20, wherein the inner tube and the outer tube form a venturi within a lumen of the outer tube.

22. The method of claim 21, wherein the venturi comprises a flared configuration of the distal end of the inner tube.

23. The method of claim 21, wherein the venturi comprises a radial narrowing of the inner surface of the outer tube from a first end toward a second end of the outer tube.

24. The method of claim 20, wherein the inner tube terminates proximal to the terminal end of the outer tube.

25. A method of delivering aerated liquid to a biological target site comprising:
inserting an aerator system into the target site, wherein the aerator system comprises an inner tube having an inner lumen, an outer tube coaxial with the inner tube, and a venturi, wherein at least a portion of the outer lumen is between the inner tube and the outer tube;
delivering a liquid through the outer lumen;
aerating the liquid, wherein aerating comprises delivering a gas through the inner lumen with a pressurized vessel; and
delivering the aerated liquid to the biological target site.

26. The method of claim 25, wherein aerating comprises concurrently delivering the gas through the inner lumen and delivering the liquid through the outer lumen.

27. The method of claim 25, wherein the aerating comprises delivering the gas through the inner lumen with the pressurized vessel independent of delivering the liquid through the outer lumen.

28. An aerator system for use in a biological target site comprising:
an inner tube;
an outer tube coaxial with the inner tube, wherein at least a portion of the outer lumen is between the inner tube and the outer tube;
a venturi element within the outer tube, wherein at least a portion of the venturi element extends beyond a distal end of the inner tube; and
a pressurized vessel connected to the inner tube.
